SEQUENCE LISTING (2 sequences)

SEQ ID NO: 1 - protein, 513 aa, Rhizoctonia solani
YVQVSPPAEHITGSQWWTDYQPVSYILTSKRGSRSQFQNMITTCKAAGVGVIADTLLNHMAGIDSGTGVAGSSFTHYNYPGIYQTQDFHHCGLESGDDIVNYSNRVEVQTCELVNLADLATDTEYVRARLATYVNDLLSLGVVGLRLDAAKHIPAGDIANILGRLSSAPYVTQEVIYGSGEPIQPSEYTGNGDVQEFRYTSALQSAFQSGGISSLNDLDSRGWVSSSNANVFVSNHDTERGGSSLNYKSGSTYTLAHIFMLAYPYGTPTVLSSYTFSDNDAGSPSSGAGSCSGSGGANGWQCQHRWTAIAGMVKWRNGVTGSVNNWVTGTNQQIAFGRGSTGFVVINNADSAWTRTFTTPLAANSYCDIISGAAGTSGTCTGASYTISGGTFTATVPARSAIALFTGAIGSGSSNSGSVSISFSVYAETTFGDNIFVSGSISQLGTWAPGSSIAMSSASYPTWTATVTLPAGTAFSYKYLRKTSSGTVVWESDPNRSATASSSGTLTLSDTWR

SEQ ID NO: 2 - DNA, 1542 bp, Rhizoctonia solani
tatgtccaggtgtctcccccagccgaacacatcaccggctcgcagtggtggactgactatcagcctgtttcgtacatacttacctcgaagcgagggagcagaagccagttccagaatatgattaccacgtgcaaggctgccggggttggtgtgattgcagatactctgttaaaccatatggccggtatcgatagtggaactggcgttgccgggtcttcgttcacgcactataactaccccggaatctaccagacccaggacttccatcactgcggcctggaatcaggtgacgacatagtcaactatagcaatcgcgtcgaagtccagacttgcgaactggtgaacctggctgaccttgctactgataccgagtatgtgcgtgctcggcttgcaacctacgtaaacgatctactgtcgttgggtgtggtaggcctacgacttgatgccgcgaaacatattcctgctggtgacattgccaacattcttggtcgtttgagttcggcaccctatgttactcaagaggttatttatggcagcggagagccgattcaaccctcggaatataccgggaatggtgatgtacaggagttccgctacacatcggcacttcagagtgctttccagagtggtgggatctcatcactgaacgatcttgacagcagaggatgggtttcttccagcaatgcgaatgtatttgtctccaaccacgataccgaacgaggcgggtcgtctcttaactacaagtctggctcgacctacacacttgctcacatcttcatgcttgcatacccttatggaactcctaccgtcttgtcttcttacaccttctcagacaacgatgctggcagtccatcgagtggtgcaggctcttgctccggatctggcggtgcgaacggatggcaatgccagcaccgctggactgcaatcgcaggaatggtcaagtggcgcaatggagtgactggcagtgtcaacaactgggtcaccggtaccaaccagcagattgcctttggtcgcgggtccactgggtttgtagttatcaacaacgcggactctgcatggaccaggacgttcacgacacccctcgctgccaactcatactgcgacataatctccggggcagcaggaactagtgggacgtgtactggagcatcgtacacgatctccggtggaacctttaccgctacagtccctgcgaggtctgcaattgctttgtttactggggcgattgggtccggatcgagcaatagcggatccgtgtctatctctttcagtgtatatgcagagaccacttttggtgacaatatcttcgtgtctggaagtatttcgcaactcggtacatgggcaccaggtagctcgatcgcaatgtcctcagcttcctatcctacatggactgccactgtgactctcccagctggtactgcgttctcttataaatatctccgtaagacctccagcggcacggtcgtatgggaatcggatcctaaccgctctgctaccgcctcctcgtccggcacattgacgcttagcgatacttggcggtga

(19) United States
(12) Patent Application Publication
(10) Pub. No.: US 2019/0136156 A1
(43) Pub. Date: May 9, 2019

(54) WASHING PERFORMANCE USING A NOVEL ALPHA-AMYLASE FROM RHIZOCTONIA SOLANI

(71) Applicant: Henkel AG & Co. KGaA, Duesseldorf (DE)

(72) Inventors: Nina Mussmann, Willich (DE); Timothy O'Connell, Landsberg am Lech (DE); Daniela Herbst, Duesseldorf (DE); Ralf G. Berger, Hannover (DE); Diana Linke, Rehburg (DE); Florian Doering, Berlin (DE)

(73) Assignee: Henkel AG & Co. KGaA, Duesseldorf (DE)

(21) Appl. No.: 16/097,532

(22) PCT Filed: May 8, 2017
(86) PCT No.: PCT/EP2017/060946

§ 371 Date: Oct. 29, 2018

(30) Foreign Application Priority Data
May 18, 2016 (DE) 10 2016 208 466.6

(51) Int. Cl. C11D 3/386; C12N 15/52; C12N 9/30; C11D 9/40

(52) U.S. Cl. CPC C11D 3/38636; C12N 15/52; C12Y 302/01001; C11D 9/40; C12N 9/242

(57) ABSTRACT

The invention relates to alpha-amylases which comprise an amino acid sequence that, over its entire length, shares at least 70% sequence identity with the amino acid sequence of SEQ ID NO. 1, and to the production and use thereof. Said type of alpha-amylases have a good cleaning performance.

### CROSS-REFERENCE TO RELATED APPLICATION

This application is a U.S. National-Stage entry under 35 U.S.C. § 371 based on International Application No. PCT/EP2017/060946, filed May 8, 2017 which was published under PCT Article 21(2) and which claims priority to German Application No. 10 2016 208 466.6, filed May 18, 2016, which are all hereby incorporated in their entirety by reference.

### TECHNICAL FIELD

The present disclosure lies in the field of enzyme technology. The present disclosure relates in particular to alpha-amylases of which the amino-acid sequence can be used, in particular with respect to use in washing and cleaning agents, to the preparation of said alpha-amylases, all reasonably similar alpha-amylases having a corresponding similar sequence according to SEQ ID NO:1 and nucleic acids which code for said alpha-amylases. The present disclosure further relates to methods and uses of said alpha-amylases, and to agents, in particular washing and cleaning agents, which contain said alpha-amylases.

### BACKGROUND

Alpha-amylases are industrially significant enzymes. The use of alpha-amylases for washing and cleaning agents is industrially established and alpha-amylases can be contained in modern, high-performance washing and cleaning agents. An alpha-amylase is an enzyme which catalyzes the hydrolysis of the inner α-(1-4)-glycosidic bonds of amylose, but which does not catalyze the cleavage of terminal or α-(1-6)-glycosidic bonds. Alpha-amylases are therefore a group of esterases (E.C. 3.2.1.1.). Alpha-amylases catalyze the cleavage of starch, glycogen and other oligosaccharides and polysaccharides which have an α-(1-4)-glycosidic bond. In this respect, alpha-amylases are effective against starch residues in laundry and catalyze the hydrolysis (endohydrolysis) of said starch residues. Alpha-amylases which have wide substrate spectra are used in particular where inhomogeneous raw materials or substrate mixtures have to be reacted, for example in washing and cleaning agents, since stains may consist of differently structured starch molecules and oligosaccharides. The alpha-amylases used in washing or cleaning agents known in the prior art are usually of microbial origin and generally come from bacteria or fungi, for example from the genera Bacillus, Pseudomonas, Acinetobacter, Micrococcus, Humicola, Trichoderma or Trichosporon, in particular Bacillus. Alpha-amylases are usually produced, according to biotechnological methods that are known per se, from suitable microorganisms, for example from transgenic hosts of the genus Bacillus or from filamentous fungi.

A particularly extensively characterized alpha-amylase is an enzyme obtained from the alkalophilic Bacillus sp. strain TS-23, which enzyme hydrolyzes at least five types of starch (Lin et al., Biotechnol Appl Biochem, 28: 61-68, 1998). The alpha-amylase from Bacillus sp. strain TS-23 has a pH optimum of 9, although it is stable over a wide pH range (i.e. pH 4.7 to 10.8). The optimal temperature of said alpha-amylase is 45° C., the enzyme also having activity at lower temperatures, for example from 15 to 20° C.

The American patent applications U.S. Pat. No. 7,407,677 B2 and U.S. Pat. No. 8,852,912 B2 also disclose specific alpha-amylases and fragments thereof for use in washing and cleaning agents.

Nevertheless, there is a need for alpha-amylase variants which have altered biochemical properties and thus provide improved performance in industrial applications.

### BRIEF SUMMARY

This disclosure provides an alpha-amylase including an amino-acid sequence which is at least about 70% identical to the amino-acid sequence shown in SEQ ID NO:1 over the total length thereof. This disclosure also provides a second alpha-amylase that is obtained from the aforementioned alpha-amylase acting as a starting molecule by employing one or more conservative amino-acid substitutions. Moreover, this disclosure provides a method for preparing an alpha-amylase including the step of providing a starting alpha-amylase which is at least about 70% identical to the amino-acid sequence shown in SEQ ID NO:1 over the total length thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has surprisingly now been found that an alpha-amylase from Rhizoctonia solani or a reasonably similar alpha-amylase (in terms of sequence identity) is particularly suitable for use in washing or cleaning agents, since it hydrolyzes a wide range of starch substrates under standard washing conditions.

The present disclosure therefore relates in a first aspect to an alpha-amylase comprising an amino-acid sequence which is at least about 70% identical to the amino-acid sequence shown in SEQ ID NO:1 over the total length thereof.

The present disclosure further relates to a method for preparing an alpha-amylase, comprising providing a starting alpha-amylase which is at least about 70% identical to the amino-acid sequence shown in SEQ ID NO:1 over the total length thereof.

An “alpha-amylase” within the meaning of the present patent application therefore covers both the alpha-amylase as such and an alpha-amylase prepared using a method as contemplated herein. All comments made with regard to the alpha-amylase therefore relate to both the alpha-amylase as a substance and the corresponding methods, in particular methods for preparing the alpha-amylase. A nucleotide sequence corresponding to the amino-acid sequence according to SEQ ID NO:1 is shown in SEQ ID NO:2.

The present disclosure further relates to the alpha-amylases as contemplated herein, to the preparation method for alpha-amylases as contemplated herein, to nucleic acids which code for these alpha-amylases, to non-human host cells which contain alpha-amylases or nucleic acids as contemplated herein, to agents, in particular washing and cleaning agents, which comprise alpha-amylases as contemplated herein, to washing and cleaning methods, and to uses of the defined alpha-amylases as contemplated herein.

The present disclosure is based on the surprising finding that an alpha-amylase as contemplated herein from Rhizoctonia solani, which alpha-amylase comprises an amino-acid sequence that is at least about 70% identical to the amino-acid sequence shown in SEQ ID NO:1, causes the hydrolysis of a wide range of starch substrates under standard washing conditions. This is particularly surprising since use in cleaning agents has not previously been described for any alpha-amylases from Rhizoctonia solani.

The alpha-amylases as contemplated herein are highly stable in washing or cleaning agents, for example with respect to surfactants and/or bleaching agents and/or with respect to temperature effects, and/or with respect to acidic or alkaline conditions and/or with respect to changes in pH and/or with respect to denaturing or oxidizing agents and/or with respect to proteolytic breakdown and/or with respect to a change in redox behaviors. Particularly preferred embodiments of the present disclosure therefore provide alpha-amylase variants that are improved in terms of performance. Advantageous embodiments of this kind of the alpha-amylases as contemplated herein therefore allow improved washing results on starch-containing stains in a wide temperature range.

An alpha-amylase as contemplated herein has enzymatic activity, i.e. it is capable of hydrolyzing starch and oligosaccharides, in particular in a washing or cleaning agent. An alpha-amylase as contemplated herein is therefore an enzyme which catalyzes the hydrolysis of α-(1-4)-glycosidic bonds in glycoside substrates and is therefore capable of cleaving starch or oligosaccharides. Furthermore, an alpha-amylase as contemplated herein is preferably a mature alpha-amylase, i.e. the catalytically active molecule without signal peptide(s) and/or propeptide(s). Unless indicated otherwise, the specified sequences also each relate to mature (processed) enzymes.

In different embodiments of the present disclosure, the alpha-amylase comprises an amino-acid sequence which is at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4%, about 99.6% and about 99.8% identical to the amino-acid sequence shown in SEQ ID NO:1 over the entire length thereof.

In further different embodiments of the present disclosure, the alpha-amylase is a free enzyme. This means that the alpha-amylase can directly act with all components of an agent and that, if the agent is a liquid agent, the alpha-amylase directly contacts the solvent of the agent as contemplated herein (e.g. water). In other embodiments, the alpha-amylase as contemplated herein in an agent can form an interaction complex with other molecules or contain a “wrapping”. In this case, a single alpha-amylase molecule or a plurality of alpha-amylase molecules can be separated from the other components of an agent by a structure surrounding said molecule(s). A separating structure of this kind can be created by, but is however not restricted to, vesicles, such as a micelle or a liposome. However, the surrounding structure can also be a virus particle, a bacterial cell or a eukaryotic cell. In various embodiments, the alpha-amylase as contemplated herein can be contained in cells of Rhizoctonia solani which express this alpha-amylase, or in cell culture supernatants of cells of this kind.

The identity of nucleic-acid or amino-acid sequences is determined by a sequence comparison. This sequence comparison is based on the BLAST algorithm (cf. for example Altschul, S. F., Gish, W., Miller, W., Myers, E. W. & Lipman, D. J. (1990) “Basic local alignment search tool.” J. Mol. Biol. 215:403-410 and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pages 3389-3402) which is established and commonly used in the prior art and is carried out, in principle, by similar series of nucleotides or amino acids in the nucleic-acid or amino-acid sequences being assigned to one another. The assignment of the relevant positions shown in a table is referred to as an “alignment”. Another algorithm available from the prior art is the FASTA algorithm. Sequence comparisons (alignments), in particular multiple sequence comparisons, are generated using computer programs. For example, the Clustal series (cf. for example Chenna et al. (2003): Multiple sequence alignment with the Clustal series of programs. Nucleic Acid Research 31, 3497-3500), T-Coffee (cf. for example Notredame et al. (2000): T-Coffee: A novel method for multiple sequence alignments. J. Mol. Biol. 302, 205-217) or programs based on these programs or algorithms are often used. Sequence comparisons (alignments) using the computer program Vector NTI® Suite 10.3 (Invitrogen Corporation, 1600 Faraday Avenue, Carlsbad, Calif., USA) with the specified standard parameters, the AlignX-Modul of which program for the sequence comparisons is based on ClustalW, are also possible.

A comparison of this kind makes it possible to confirm the similarity between the compared sequences. This similarity is usually expressed in percent identity, i.e. the percentage of identical nucleotides or amino-acid residues at the same positions or at positions that correspond to one another in an alignment. In amino-acid sequences, the broader concept of “homology” factors in conserved amino-acid exchanges, i.e. amino acids having similar chemical activity, since these usually have similar chemical activities within the protein. Therefore, the similarity between the compared sequences can also be expressed in percent homology or percent similarity. Identity and/or homology values may apply to the entirety of the polypeptides or genes or only to individual segments. Homologous or identical segments of different nucleic-acid or amino-acid sequences are therefore defined by matches in the sequences. Segments of this kind often have identical functions. Said segments may be small and only comprise a few nucleotides or amino acids. Segments that are this small often perform functions that are essential to the overall activity of the protein. Therefore, it may be expedient for sequence matches to only relate to individual, optionally small, segments. Unless indicated otherwise, identity or homology values in the present application refer to the total length of the nucleic-acid sequence or amino-acid sequence specified in each case.

In the context of the present disclosure, if it is stated that an amino-acid position corresponds to a numerically identified position in SEQ ID NO:1, this means that the corresponding position is assigned to the numerically identified position in SEQ ID NO:1 in an alignment as defined above.

In a further embodiment of the present disclosure, the alpha-amylase cleaning performance is not significantly reduced by comparison with an alpha-amylase which has an amino-acid sequence that corresponds to the amino-acid sequence shown in SEQ ID NO:1, i.e. said alpha-amylase has at least about 70%, about 75%, about 80%, about 85%, about 90%, about 95% of the reference washing performance. The cleaning performance can be determined in a washing system which contains a washing agent in a dosage of between from about 4.5 and about 7.0 grams per liter of washing liquor, and the alpha-amylase, the alpha-amylases to be compared being used in the same concentration (based on the active protein) and the cleaning performance with respect to a stain on cotton is determined by measuring the extent to which the washed textiles have been cleaned. For example, the washing process can be carried out for about 60 minutes at a temperature of about 40° C., and the water can have a water hardness of between from about 5° and about 25°, preferably from about 10° and about 20°, more preferably from about 13° and about 17°, and even more preferably from about 15.5° and about 16.5° (German degree of hardness). The concentration of the alpha-amylase in the washing agent intended for this washing system is from about 0.001 to about 1 wt. %, preferably from about 0.001 to about 0.1 wt. %, and even more preferably from about 0.01 to about 0.06 wt. %, based on the purified active protein.

A preferred liquid washing agent for a washing system of this kind is composed as follows (all amounts are given in percent by weight): about 7% of alkyl benzene sulfonic acid, about 9% of anionic surfactants, about 4% of C₁₂-C₁₈ Na salts of fatty acids, about 7% of non-ionic surfactants, about 0.7% of phosphonates, about 3.2% of citric acid, about 3.0% of NaOH, about 0.04% of defoamer, about 5.7% of 1,2-propanediol, about 0.1% of preservatives, about 2% of ethanol, about 0.2% of dye-transfer-inhibitors, and the remaining percentage of demineralized water. The dosage of the liquid washing agent is preferably between from about 4.5 and about 6.0 grams per liter of washing liquor, for example from about 4.7, 4.9 or about 5.9 grams per liter of washing liquor. Washing is preferably carried out within a pH range of between from about pH 7.5 and about pH 10.5, preferably between from about pH 7.5 and about pH 9.

Within the scope of the present disclosure, the cleaning performance is determined at about 40° C. using a liquid washing agent as specified above, the washing process preferably being carried out for about 60 minutes.

The degree of whiteness, i.e. the lightening of the stains, is determined using optical measurement methods, preferably photometrically, as a measure of cleaning performance. A device suitable for this purpose is the spectrometer Minolta CM508d, for example. The devices used for the measurement are usually calibrated, in advance, against a white standard, preferably a white standard that is supplied therewith.

Each alpha-amylase being applied in an identical manner in terms of activity ensures that the relevant enzymatic properties, i.e. for example cleaning performance on particular stains, are compared even if there is some kind of divergence in the ratio of active substance to overall protein (the values for specific activity). In general, low specific activity can be compensated for by adding a larger amount of protein. Moreover, the enzymes to be tested can also be used in the same substance amount or amount by weight, if the enzymes to be tested have a different affinity to the test substrate in an activity test. The expression “same substance amount” relates, in this context, to using the same mols of the enzymes to be tested. The expression “same amount by weight” relates to using the same weight of the enzymes to be tested.

The alpha-amylase activity is determined in a manner that is routine in the art, specifically preferably by employing an optical measurement method, more preferably a photometric method. The test suitable for this purpose comprises the alpha-amylase-dependent cleavage of the substrate para-nitrophenyl maltoheptaoside. This is cleaved into para-nitrophenyl oligosaccharide by the alpha-amylase. The para-nitrophenyl oligosaccharide is in turn catalyzed by the enzymes glucoamylase and alpha-glucosidase to form glucose and para-nitrophenol. The presence of para-nitrophenol can be determined using a photometer, e.g. the Tecan Sunrise Device and XFLUOR software, at about 405 nm, and it is thus possible to draw a conclusion on the enzymatic activity of the alpha-amylase.

The protein concentration can be determined using known methods, for example the BCA method (bicinchoninic acid; 2,2′-biquinolyl-4,4′-dicarboxylic acid) or the Biuret method. (A. G. Gornall, C. S. Bardawill and M. M. David, J. Biol. Chem., 177 (1948), pages 751-766). The active protein concentration can be determined, in this respect, by titrating the active centers using a suitable irreversible inhibitor and determining the residual activity (cf. M. Bender et al., J. Am. Chem. Soc. 88, 24 (1966), pages 5890-5913).

Proteins can be combined to form groups of immunologically related proteins by employing the reaction with an antiserum or a particular antibody. Those which belong to a group of this kind are exemplified in that they have the same antigenic determinants which are detected by an antibody. They are therefore structurally so similar to one another that they are detected by an antiserum or particular antibodies. A further subject of the present disclosure is therefore alpha-amylases which are exemplified in that they have at least one and, in order of increasing preference, two, three or four antigenic determinants which match an alpha-amylase as contemplated herein. Alpha-amylases of this kind are structurally so similar to the alpha-amylases as contemplated herein as a result of their immunological matches that a similar function can also be assumed.

By comparison with the alpha-amylases described in SEQ ID NO:1, alpha-amylases as contemplated herein can have further amino-acid alterations, in particular amino-acid substitutions, insertions or deletions. Alpha-amylases of this kind are developed, for example, by targeted genetic alteration, i.e. by mutagenesis methods, and optimized for particular uses or in respect of specific properties (for example in respect of their catalytic activity, stability, etc.). Furthermore, nucleic acids as contemplated herein can be incorporated in recombination approaches and are thus used to produce completely new types of alpha-amylases or other polypeptides.

The aim is to introduce targeted mutations, such as substitutions, insertions or deletions, into known molecules, in order to improve the cleaning performance of enzymes as contemplated herein, for example. For this purpose, in particular the surface charges and/or isoelectric point of the molecules, and thus their interactions with the substrate, can be altered. For example, the net charge of the enzymes can be changed in order to thereby influence the substrate binding, in particular for use in washing and cleaning agents. Alternatively or in addition, the stability of the alpha-amylase can be increased further still, and thus the cleaning performance thereof can be improved, by one or more appropriate mutations. Advantageous properties of individual mutations, e.g. individual substitutions, may complement one another. An alpha-amylase that has already been optimized in terms of particular properties, for example in terms of the activity thereof and/or the tolerance thereof with respect to the substrate spectrum, can therefore also be developed within the scope of the present disclosure.

In order to describe substitutions that affect exactly one amino-acid position (amino-acid exchanges), the following convention is used: the internationally conventional single-letter code of the naturally present amino acid is given first, and then the associated sequence position, and finally the amino acid that has been added. Several exchanges within the same polypeptide chain are separated from one another by slashes. For insertions, additional amino acids are indicated after the sequence position. For deletions, the amino acid that has been removed is replaced with a symbol, for example a star or a dash, or a Δ is put before the corresponding position. For example, N62Q denotes the substitution of asparagine at position 62 by glutamine, N62AQ denotes the insertion of alanine after the amino acid asparagine at position 62, and N62* or ΔN62 denotes the deletion of asparagine from position 62. This nomenclature is known to a person skilled in the art of enzyme technology.

Therefore, the present disclosure further relates to an alpha-amylase which can be obtained from an alpha-amylase as described above acting as a starting molecule by employing one or more conservative amino-acid substitutions. The term “conservative amino-acid substitutions” means the exchange (substitution) of an amino-acid residue with another amino-acid residue, this exchange not resulting in a change in the polarity or charge at the position of the exchanged amino acid, e.g. the exchange of a nonpolar amino-acid residue with another nonpolar amino-acid residue. Within the scope of the present disclosure, conservative amino-acid substitutions include for example: G=A=S, I=V=L=M, D=E, N=Q, K=R, Y=F, S=T, G=A=I=V=L=M=Y=F=W=P=S=T.

Alternatively or in addition, the alpha-amylase can be obtained from an alpha-amylase as contemplated herein as a starting molecule by employing fragmentation, deletion, insertion or substitution mutagenesis, and comprises an amino-acid sequence which matches the starting molecule over a length of at least about 360, about 370, about 380, about 390, about 400, about 410, about 420, about 430, about 440, about 450, about 460, about 470, about 480, about 490, about 500, about 510 or about 513 interconnected amino acids.

It is thus possible, for example, for individual amino acids to be deleted from the enzyme termini or loops, without this resulting in the endohydrolytic activity being lost or reduced. Furthermore, by employing fragmentation, deletion, insertion or substitution mutagenesis of this kind, the allergenicity of relevant enzymes, for example, can also be reduced and thus the usability thereof can be improved overall. The enzymes advantageously still have their endohydrolytic activity even after the mutagenesis, i.e. the endohydrolytic activity thereof corresponds at least to that of the starting enzyme, i.e. in a preferred embodiment, the endohydrolytic activity is at least about 80%, preferably at least about 90%, of the activity of the starting enzyme. Other substitutions can also have advantageous effects. It is possible to exchange a single amino acid or several interconnected amino acids with other amino acids.

In this case, the other amino-acid positions are defined by an alignment of the amino-acid sequence of an alpha-amylase as contemplated herein with the amino-acid sequence of the alpha-amylase from Rhizoctonia solani, as shown in SEQ ID NO:1. Furthermore, the assignment of the positions is determined by the mature protein. In particular, this assignment is also used if the amino-acid sequence of an alpha-amylase as contemplated herein has a higher number of amino-acid residues than the alpha-amylase from Rhizoctonia solani according to SEQ ID NO:1. Proceeding from the mentioned positions in the amino-acid sequence of the alpha-amylase from Rhizoctonia solani, the alteration positions in an alpha-amylase as contemplated herein are those which are precisely assigned to these positions in an alignment.

Further confirmation of the correct assignment of the amino acids to be altered, i.e. in particular of the functional correspondence thereof, can be provided by comparison tests during which the two positions assigned to one another on the basis of an alignment are altered in the same way in the two alpha-amylases being compared with one another and it is observed whether the enzymatic activity is altered in the same way in the two alpha-amylases. If, for example, an amino-acid exchange at a particular position of the alpha-amylase from Rhizoctonia solani according to SEQ ID NO:1 is associated with a change in an enzymatic parameter, for example with the increase in the KM value, and if a corresponding change in the enzymatic parameter, thus for example also an increase in the KM value, is observed in an alpha-amylase variant as contemplated herein of which the amino-acid exchange was achieved by the same added amino acid, this is considered to be confirmation of the correct assignment.

All elements specified can also be applied to the methods as contemplated herein for preparing an alpha-amylase. A method as contemplated herein therefore further comprises one or more of the following method steps:

a. introducing one or more conservative amino-acid substitutions into a starting alpha-amylase according to SEQ ID NO:1;
b. altering the amino acid sequence by employing fragmentation, deletion, insertion or substitution mutagenesis such that the alpha-amylase has an amino acid sequence which matches the starting molecule over a length of at least about 360, about 370, about 380, about 390, about 400, about 410, about 420, about 430, about 440, about 450, about 460, about 470, about 480, about 490, about 500, about 510, or about 513 interconnected amino acids.

All comments made also apply to the methods as contemplated herein.

In further embodiments of the present disclosure, the alpha-amylase as contemplated herein or the alpha-amylase prepared using a method as contemplated herein is still at least about 70%, about 71%, about 72% about, 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4%, about 99.6% or about 99.8% identical to the amino-acid sequence shown in SEQ ID NO:1 over the entire length thereof.

The present disclosure also relates to an alpha-amylase as described above which is additionally stabilized, in particular by employing one or more mutations, for example substitutions, or by being coupled to a polymer. Increasing stability during storage and/or during use, for example during the washing process, leads to the enzymatic activity being maintained for longer and thus to the cleaning performance being improved. In principle, all stabilizing possibilities that are expedient and/or described in the prior art can be considered for this. Stabilizations which are achieved by employing mutations of the enzyme itself are preferred, since stabilizations of this kind do not require any further working steps after the enzyme has been obtained. Examples of sequence alterations suitable for this purpose have been mentioned above. Further suitable sequence alterations are known from the prior art.

Possibilities for stabilization include for example:
protecting against the influence of denaturing agents, such as surfactants, by employing mutations which cause the amino-acid sequence to be altered on or at the surface of the protein;
exchanging amino acids which are close to the N-terminus with amino acids which are assumed to come into contact with the rest of the molecule by employing non-covalent interactions, and thus contribute to maintaining the globular structure.

Preferred embodiments are those in which the enzyme is stabilized in several different ways, since several stabilizing mutations have a cumulative or synergistic effect.

The present disclosure also relates to an alpha-amylase as described above which has at least one chemical modification. An alpha-amylase that is altered in this way is referred to as a “derivative”, i.e. the alpha-amylase is derivatized.

Within the meaning of the present application, “derivatives” are therefore understood to mean proteins of which the pure amino-acid chain has been chemically modified. Derivatizations of this kind can be carried out in vivo, for example, by the host cell which expresses the protein. In this respect, couplings to low-molecular-weight compounds, such as lipids or oligosaccharides, are of particular importance. However, derivatizations may also be carried out in vitro, for example by the chemical conversion of a side chain of an amino acid or by covalent bonding of another compound to the protein. For example, it is possible to couple amines to carboxyl groups of an enzyme in order to change the isoelectric point. This other compound may also be another protein which is bound to a protein as contemplated herein via bifunctional chemical compounds, for example. Derivatization is also understood to mean covalent bonding to a macromolecular carrier, or non-covalent confinement in suitable macromolecular cage structures. Derivatizations can, for example, influence the substrate specificity or the bond strength to the substrate or cause temporary inhibition of enzymatic activity, if the coupled substance is an inhibitor. This can be expedient in terms of the period of storage, for example. Modifications of this kind can also influence stability or enzymatic activity. They can also be used to reduce the allergenicity and/or immunogenicity of the protein and to thus increase the skin compatibility thereof. For example, couplings to macromolecular compounds, for example polyethylene glycol, can improve the protein in terms of stability and/or skin compatibility.

In the broadest sense, derivatives of a protein as contemplated herein can be understood to also include preparations of these proteins. Depending on how a protein is obtained, recovered or prepared, said protein can be accompanied by a wide range of other substances, for example from the culture of the microorganisms that produce it. A protein may also have been deliberately mixed with other substances in order to increase its storage stability, for example. Therefore, the present disclosure also covers all preparations of a protein as contemplated herein. This is still true irrespective of whether or not this enzymatic activity actually develops in a particular preparation. This is because it may be desirable for the protein to not have any activity or to only have low activity when being stored, and for the enzymatic function to only develop once the protein is in use. This can be controlled, for example, by appropriate accompanying substances. In particular, in this respect, it is possible to jointly prepare alpha-amylases and specific inhibitors.

With regard to all above-described alpha-amylases or alpha-amylase variants and/or derivatives, within the scope of the present disclosure, alpha-amylases, alpha-amylase variants and/or derivatives of which the catalytic activity and/or the substrate tolerance corresponds to that of the alpha-amylase according to SEQ ID NO:1 are particularly preferred, the catalytic activity and the substrate tolerance being determined as described above.

The present disclosure also relates to a nucleic acid which codes for an alpha-amylase as contemplated herein, and to a vector containing a nucleic acid of this kind, in particular a cloning vector or an expression vector. In preferred embodiments, the nucleic acid is a nucleic acid according to SEQ ID NO:2. Accordingly, a particularly preferred vector as contemplated herein is a vector which comprises a nucleic acid according to SEQ ID NO:2.

These may be DNA or RNA molecules. They may be present as a single strand, as a single strand that is complementary to the first single strand, or as a double strand. In the case of DNA molecules in particular, the sequences of the two complementary strands should be taken into account in all three possible reading frames. It should also be noted that different codons, i.e. base triplets, can code for the same amino acids, such that a particular amino-acid sequence can be coded for by several different nucleic acids. Owing to this degeneracy of the genetic code, all nucleic-acid sequences which can code for one of the above-described alpha-amylases are included in this subject of the present disclosure. A person skilled in the art is able to identify these nucleic-acid sequences with absolute certainty since, despite the degeneracy of the genetic code, defined amino acids can be assigned to individual codons. Therefore, proceeding from an amino-acid sequence, a person skilled in the art can easily identify nucleic acids which code for said amino-acid sequence. Furthermore, in nucleic acids as contemplated herein, one or more codons can be replaced by synonymous codons. This aspect relates in particular to the heterologous expression of the enzymes as contemplated herein. Therefore, each organism, for example a host cell of a production strain, has a particular codon usage. “Codon usage” is understood to mean the translation of the genetic code into amino acids by employing the relevant organism. Bottlenecks can occur in protein biosynthesis if the codons on the nucleic acid are accompanied by a comparatively low number of charged tRNA molecules in the organism. Although coding for the same amino acid, this leads to a codon being translated less efficiently in the organism than a synonymous codon which codes for the same amino acid. Owing to the presence of a higher number of tRNA molecules for the synonymous codon, said codon can be translated more efficiently in the organism.

Using methods which are currently generally known, such as chemical synthesis or the polymerase chain reaction (PCR), in conjunction with molecular biological and/or protein chemical standard methods, it is possible for a person skilled in the art, on the basis of known DNA and/or amino acid sequences, to produce the corresponding nucleic acids and even complete genes. Methods of this kind are known from, for example, Sambrook, J., Fritsch, E. F. and Maniatis, T. 2001. Molecular cloning: a laboratory manual, 3rd Edition Cold Spring Laboratory Press.

Within the meaning of the present disclosure, vectors are understood to mean elements which include nucleic acids and which include a nucleic acid as contemplated herein as an exemplified nucleic-acid range. Vectors allow establishment of this nucleic acid in a species or a cell line over multiple generations or cell divisions as a stable genetic element. Vectors are specific plasmids, i.e. circular genetic elements, in particular for use in bacteria. Within the scope of the present disclosure, a nucleic acid as contemplated herein is cloned in a vector. These may include vectors, for example, which originate from bacterial plasmids, from viruses, or from bacteriophages, or predominantly synthetic vectors or plasmids having elements of various origins. Using the further genetic elements which are present in each case, vectors are able to become established as stable units in the host cells in question over several generations. They may be present as separate units outside of a chromosome or be integrated in a chromosome or chromosomal DNA.

Expression vectors have nucleic-acid sequences which enable them to replicate in the host cells, preferably microorganisms, particularly preferably bacteria, which contain them and to express therein a contained nucleic acid. The expression is influenced, in particular, by promoter(s) which regulate the transcription. In principle, the expression can be carried out by the natural promoter which is originally located in front of the nucleic acid to be expressed, by a promoter of the host cell provided on the expression vector, or by a modified or completely different promoter of another organism or another host cell. In the present case, at least one promoter is provided for the expression of a nucleic acid as contemplated herein and is used for the expression thereof. Expression vectors can also be regulated, for example by changing the culturing conditions, by reaching a particular cell density in the host cells containing said vectors, or by adding particular substances, in particular activators for gene expression. An example of a substance of this kind is the galactose derivative isopropyl-β-D-thiogalactopyranoside (IPTG) which is used as an activator for the bacterial lactose operon (lac operon). Unlike in expression vectors, the contained nucleic acid in cloning vectors is not expressed.

The present disclosure also relates to a non-human host cell containing a nucleic acid as contemplated herein or a vector as contemplated herein, or containing an alpha-amylase as contemplated herein, in particular a non-human host cell which secretes the alpha-amylase into the medium surrounding the host cell. A nucleic acid as contemplated herein or a vector as contemplated herein is preferably transformed into a microorganism which then constitutes a host cell as contemplated herein. Alternatively, individual components, i.e. nucleic-acid parts or fragments of a nucleic acid as contemplated herein, can be introduced into a host cell such that the resulting host cell contains a nucleic acid as contemplated herein or a vector as contemplated herein. This procedure is particularly suitable if the host cell already contains one or more components of a nucleic acid as contemplated herein or of a vector as contemplated herein, and the additional components are then added accordingly. Methods for transforming cells are established in the prior art and are sufficiently known to a person skilled in the art. In principle, all cells, i.e. prokaryotic or eukaryotic cells, are suitable as host cells. Preferred host cells are those which may be advantageously managed genetically, which involves, for example, transformation using the nucleic acid or the vector and stable establishment thereof, for example unicellular fungi or bacteria. In addition, preferred host cells are distinguished by good microbiological and biotechnological manageability. This relates, for example, to ease of culturing, high growth rates, low demands on fermentation media, and good production and secretion rates for foreign proteins. Preferred host cells as contemplated herein secrete the (transgenically) expressed protein into the medium surrounding the host cells. Furthermore, the alpha-amylases can be modified, following preparation, by the cells that produced them, for example by the attachment of sugar molecules, by formylations, by aminations, etc. Post-translational modifications of this kind can influence the alpha-amylase in terms of its function.

Those host cells of which the activity can be regulated due to genetic regulation elements which are provided on the vector, for example, but which may also be present in these cells from the outset, represent other preferred embodiments. These host cells may be induced to express, for example by the controlled addition of chemical compounds which are used as activators, by changing the culturing conditions, or upon reaching a particular cell density. This provides for cost-effective production of the proteins as contemplated herein. An example of a compound of this kind is IPTG, as described above.

Prokaryotic or bacterial cells are preferred host cells. Bacteria are distinguished by short generation times and low demands on the culturing conditions. Cost-effective culturing methods or preparation methods can thereby be established. Furthermore, a person skilled in the art has a vast pool of experience with regard to bacteria in fermentation technology. Gram-negative or gram-positive bacteria may be suitable for specific production for a wide variety of reasons, which should be determined by experiment in any given case, for example nutrient sources, product formation rate, time constraints, etc.

In the case of gram-negative bacteria, such as Escherichia coli, numerous proteins are secreted into the periplasmatic space, i.e. the compartment between the two membranes which enclose the cells. This may be advantageous for specific applications. Furthermore, gram-negative bacteria may also be formed such that they secrete the expressed proteins not only into the periplasmatic space, but also into the medium surrounding the bacterium. By contrast, gram-positive bacteria, for example Bacilli or Actinomycetes or other representatives of the Actinomycetales, have no outer membrane, and therefore secreted proteins are released directly into the medium surrounding the bacteria, generally the nutrient medium, from which the expressed proteins may be purified. They may be isolated directly from the medium or processed further. Moreover, gram-positive bacteria are related to or identical to most origin organisms for industrially significant enzymes and they themselves usually form comparable enzymes, such that they have a similar codon usage and the protein synthesis apparatus thereof is naturally aligned accordingly.

Host cells as contemplated herein may be altered in terms of their requirements for culture conditions, may have different or additional selection markers, or may express different or additional proteins. These host cells may be in particular host cells that express a plurality of proteins or enzymes transgenically.

The present disclosure can be used, in principle, for all microorganisms, in particular for all fermentable microorganisms, particularly preferably for those from the Bacillus genus, and leads to it being possible to prepare proteins as contemplated herein by using microorganisms of this kind. Microorganisms of this kind then constitute host cells within the meaning of the present disclosure.

In a further embodiment of the present disclosure, the host cell is a bacterium, preferably a bacterium selected from the group of the genera of Escherichia, Klebsiella, Bacillus, Staphylococcus, Corynebacterium, Arthrobacter, Streptomyces, Stenotrophomonas and Pseudomonas, more preferably a bacterium selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell may also be a eukaryotic cell which has a nucleus. Therefore, the present disclosure further relates to a host cell which has a nucleus. Unlike prokaryotic cells, eukaryotic cells are able to modify the formed protein post-translationally. Examples of eukaryotic cells are fungi such as actinomycetes or yeasts such as Saccharomyces or Kluyveromyces. This may be particularly advantageous, for example, if, in the context of their synthesis, the proteins are intended to undergo specific modifications which systems of this kind allow. The modifications which are carried out by eukaryotic systems, particularly in the context of protein synthesis, include, for example, the binding of low-molecular-weight compounds such as membrane anchors or oligosaccharides. Oligosaccharide modifications of this kind may be desirable, for example, as a way to reduce the allergenicity of an expressed protein. A co-expression with the enzymes formed naturally by cells of this kind, such as cellulases, can also be advantageous. Furthermore, thermophilic fungal expression systems, for example, may be particularly suitable for expressing temperature-resistant proteins or variants. In preferred embodiments of the present disclosure, the host cell is a basidiomycete cell. In more preferred embodiments, the host cell is a Rhizoctonia solani cell.

The host cells as contemplated herein are cultured and fermented in a conventional manner, for example in batch or continuous systems. In the first case, a suitable nutrient medium is inoculated with the host cells, and the product is harvested from the medium after a period of time that can be determined by experiment. Continuous fermentation is distinguished by the achievement of a steady state in which, over a comparatively long period of time, some cells die, but also regenerate, and at the same time, the formed protein can be removed from the medium.

Host cells as contemplated herein are preferably used in order to prepare alpha-amylases as contemplated herein. Therefore, the present disclosure also relates to a method for preparing an alpha-amylase, comprising:
culturing a host cell as contemplated herein, and
isolating the alpha-amylase from the culture medium or from the host cell.

This subject of the present disclosure preferably includes fermentation methods. Fermentation methods are known per se from the prior art, and constitute the actual large-scale production step, generally followed by a suitable method for purifying the prepared product, for example the alpha-amylase as contemplated herein. All fermentation methods which are based on a corresponding method for preparing an alpha-amylase as contemplated herein constitute embodiments of this subject of the present disclosure.

Fermentation methods which are exemplified in that the fermentation is carried out via an inflow strategy are considered in particular. Here, the media components that are consumed by the continuous culturing are fed in. Significant increases both in the cell density and in the cell mass or dry mass, and/or in particular in the activity of the alpha-amylase of interest, can be achieved in this way. Furthermore, the fermentation may also be designed in such a way that undesirable metabolic products are filtered out, or neutralized by adding a buffer or appropriate counterions.

The prepared alpha-amylase can be harvested from the fermentation medium. A fermentation method of this kind is preferred over isolation of the alpha-amylase from the host cell, i.e. product recovery from the cell mass (dry mass); however, said method requires that suitable host cells or one or more suitable secretion markers or mechanisms and/or transport systems be provided, so that the host cells secrete the alpha-amylase into the fermentation medium. Alternatively, without secretion, the alpha-amylase can be isolated from the host cell, i.e. separated from the cell mass, for example by precipitation with ammonium sulfate or ethanol, or by chromatographic purification.

All aforementioned elements can be combined to form methods for preparing alpha-amylases as contemplated herein.

The present disclosure also relates to an agent which contains an alpha-amylase as contemplated herein, as described above. The agent is preferably a washing or cleaning agent.

This covers all conceivable types of washing or cleaning agents, including both concentrates and agents to be used in undiluted form, for use on a commercial scale, in washing machines or for washing or cleaning by hand. These agents include, for example, washing agents for textiles, carpets or natural fibers for which the term “washing agent” is used. These also include, for example, dishwashing detergents for dishwashers or manual dishwashing detergents or cleaners for hard surfaces, such as metal, glass, porcelain, ceramics, tiles, stone, coated surfaces, plastics materials, wood or leather for which the term “cleaning agent” is used, i.e. in addition to manual and automatic dishwashing detergents, for example also abrasive cleaners, glass cleaners, WC rim blocks, etc. Within the scope of the present disclosure, the washing and cleaning agents also include auxiliary washing agents, which are added to the actual washing agent when washing textiles manually or using a machine in order to achieve an additional effect. Furthermore, within the scope of the present disclosure, washing and cleaning agents also include textile pre-treatment and post-treatment agents, i.e. agents with which the piece of laundry comes into contact before it is actually washed, for example in order to loosen stubborn dirt, and also agents which impart other desirable properties to the laundry, for example softness to touch, crease resistance or low static charge, in a step that comes after the actual textile washing process. The agents mentioned last include, inter alia, softeners.

The washing or cleaning agents as contemplated herein, which may be present in the form of powdered solids, compressed particles, homogeneous solutions or suspensions, can contain, in addition to an alpha-amylase as contemplated herein, all known ingredients that are common in agents of this kind, at least one further ingredient preferably being present in the agent. The agents as contemplated herein may contain surfactants, builders, peroxygen compounds or bleach activators, in particular. They may also contain water-miscible organic solvents, further enzymes, sequestering agents, electrolytes, pH regulators and/or further auxiliaries such as optical brighteners, graying inhibitors, foam regulators, and dyes and fragrances, and combinations thereof.

In particular, a combination of an alpha-amylase as contemplated herein with one or more further ingredient(s) of the agent is advantageous, since an agent of this kind has improved cleaning performance in preferred embodiments as contemplated herein on account of synergies obtained thereby. In particular, such synergy can be achieved by the combination of an alpha-amylase as contemplated herein with a surfactant and/or a builder and/or a peroxygen compound and/or a bleach activator.

Advantageous ingredients of agents as contemplated herein are disclosed in international patent application WO 2009/121725, starting on the penultimate paragraph of page 5 and ending on page 13 after the second paragraph. Reference is made explicitly to this disclosure and the content thereof is incorporated in the present patent application.

An agent as contemplated herein advantageously contains the alpha-amylase in an amount of from about 2 μg to about 20 mg, preferably from about 5 μg to about 17.5 mg, particularly preferably from about 20 μg to about 15 mg, and very particularly preferably from about 50 μg to about 10 mg per g of the agent. Furthermore, the agent as contemplated herein can advantageously contain the alpha-amylase in an amount of from about 0.00005 to about 15 wt. %, preferably from about 0.0001 to about 5 wt. %, and particularly preferably from about 0.001 to about 1 wt. %, based on the active enzyme. Furthermore, the alpha-amylase contained in the agent and/or further ingredients of the agent may be encapsulated in a substance that is impermeable to the enzyme at room temperature or in the absence of water, which substance becomes permeable to the enzyme under use conditions of the agent. Such an embodiment of the present disclosure is thus exemplified in that the alpha-amylase is encapsulated in a substance that is impermeable to the alpha-amylase at room temperature or in the absence of water. Furthermore, the washing or cleaning agent itself can also be packaged in a container, preferably an airtight container, from which it is released shortly before use or during the washing process.

In other embodiments of the present disclosure, the agent
a) is present in solid form, in particular as a flowable powder having a bulk density of from about 300 g/l to about 1200 g/1, in particular from about 500 g/l to about 900 g/1, or
b) is present in pasty or liquid form, and/or
c) is present in gel or pouch form, and/or
d) is present as a single-component system, or
e) is divided into a plurality of components.

These embodiments of the present disclosure cover all solid, powder, liquid, gel or paste dosage forms of agents as contemplated herein that may optionally also include a plurality of phases, and may be present in compressed or uncompressed form. The agent may be present in the form of a flowable powder, in particular having a bulk density of from about 300 g/l to about 1200 g/1, more particularly from about 500 g/l to about 900 g/l or from about 600 g/l to about 850 g/1. The solid dosage forms of the agent also include extrudates, granules, tablets or pouches. Alternatively, the agent may also be a liquid, gel or paste, for example in the form of a non-aqueous liquid washing agent or a non-aqueous paste or in the form of an aqueous liquid washing agent or a water-containing paste. Furthermore, the agent may be present as a single-component system. Agents of this kind consist of one phase. Alternatively, an agent can also consist of a plurality of phases. An agent of this kind is therefore divided into a plurality of components.

Washing or cleaning agents as contemplated herein may only contain an alpha-amylase. Alternatively, they may also contain further hydrolytic enzymes or other enzymes in a concentration that is expedient in terms of the effectiveness of the agent. Another embodiment of the present disclosure thus relates to agents which also comprise one or more further enzymes. All enzymes which can develop catalytic activity in the agent as contemplated herein, in particular a protease, lipase, cellulase, hemicellulase, mannanase, tannase, xylanase, xanthanase, xyloglucanase, β-glucosidase, pectinase, carrageenase, perhydrolase, oxidase, oxidoreductase or other alpha-amylases that are different from the alpha-amylases as contemplated herein, and mixtures thereof, can preferably be used as further enzymes. Further enzymes are contained in the agent advantageously in an amount of from 1×10⁻⁸ to about 5 wt. % in each case, based on the active protein. Each further enzyme is contained in agents as contemplated herein in an amount of, in order of increasing preference, from 1×10⁻⁷ to about 3 wt. %, from about 0.00001 to about 1 wt. %, from about 0.00005 to about 0.5 wt. %, from about 0.0001 to about 0.1 wt. %, and most particularly preferably from about 0.0001 to about 0.05 wt. %, based on the active protein. The enzymes particularly preferably have synergistic cleaning performances with respect to particular stains or marks, i.e. the enzymes contained in the agent composition assist one another in terms of the cleaning performance thereof. Very particularly preferably, such synergy exists between the alpha-amylase contained as contemplated herein and a further enzyme of an agent as contemplated herein, in particular between the stated alpha-amylase and a lipase and/or a protease and/or a mannanase and/or a cellulase and/or a pectinase. Synergistic effects can occur not only between different enzymes, but also between one or more enzymes and other ingredients of the agent as contemplated herein.

The enzymes to be used in the cleaning agents described herein can also be formulated together with accompanying substances, for example from fermentation. In liquid formulations, the enzymes are preferably used as an enzyme liquid formulation or enzyme liquid formulations.

The enzymes are usually not made available in the form of the pure protein, but rather in the form of stabilized, storable and transportable preparations. These ready-made preparations include, for example, the solid preparations obtained through granulation, extrusion, or lyophilization or, particularly in the case of liquid or gel-type agents, solutions of the enzymes, advantageously maximally concentrated, low-moisture, and/or supplemented with stabilizers or other adjuvants.

Alternatively, the enzymes can also be encapsulated for both the solid and liquid dosage form, for example through spray-drying or extrusion of the enzyme solution together with a preferably natural polymer, or in the form of capsules, for example those in which the enzymes are enclosed in a set gel, or in those of the core-shell type in which an enzyme-containing core is coated with a water-, air-, and/or chemical-impermeable protective layer. In the case of overlaid layers, other active ingredients, such as stabilizers, emulsifiers, pigments, bleaching agents, or dyes, can be additionally applied. Capsules of this kind are applied using inherently known methods, for example through shaking or roll granulation or in fluidized bed processes. Such granulates are advantageously low in dust, for example due to the application of polymeric film-formers, and stable in storage due to the coating.

The enzymes can also be introduced into water-soluble films. A film of this kind makes it possible to release the enzymes after contact with water. As used here, “water-soluble” relates to a film structure which is preferably completely water-soluble. However, the term “water-soluble” also includes: films which are substantially water-soluble but have relatively small amounts of a material that is not water-soluble in the film structure; films comprising materials that are only water-soluble in relatively high water temperatures or only under restricted pH conditions; and films which comprise a relatively thin layer of water-insoluble material. A film of this kind preferably includes (completely or partially hydrolyzed) polyvinyl alcohol (PVA). However, the film can also contain, exclusively or in addition to PVA, acid-acrylate copolymers, preferably methacrylic acid-ethyl acrylate copolymer, as can be obtained from Beiland as GBC 2580 and 2600, styrene maleic anhydride copolymer (SMA) (available as Scripset (trade name) from Monsanto); ethylene-acrylic acid copolymer (EAA) or ethylene-methacrylic acid copolymer (EMMA) which is neutralized by metal salt, known as lonomer (available from du Pont), the acid content of EAA or EMMA being at least approximately 20 mol. %; polyether block amide copolymer; polyhydroxyvaleric acid (available as Biopol (trade name) resins from Imperial Chemical Industries); polyethylene oxide; water-soluble polyesters or copolyesters; polyethyloxazoline (PEOX 200 from Dow); and water-soluble polyurethane.

Moreover, it is possible to formulate two or more enzymes together, so that a single granulate has several enzyme activities.

The present disclosure further relates to a method for cleaning textiles or hard surfaces, exemplified in that an agent as contemplated herein is used in at least one method step, or in that an alpha-amylase as contemplated herein becomes catalytically active in at least one method step, in particular such that the alpha-amylase is used in an amount of from about 40 μg to about 4 g, preferably from about 50 μg to about 3 g, particularly preferably from about 100 μg to about 2 g, and very particularly preferably from about 200 μg to about 1 g.

In various embodiments, the above-described method is distinguished in that the alpha-amylase is used at a temperature of from about 0 to about 100° C., preferably from about 0 to about 60° C., more preferably from about 20 to about 45° C., and most preferably at a temperature of about 40° C.

These embodiments include both manual and automatic methods, automatic methods being preferred. Methods for cleaning textiles are generally distinguished in that various substances that have a cleaning effect are applied to the item to be cleaned in a plurality of method steps and washed off after the contact time, or in that the item to be cleaned is treated with a washing agent or a solution or dilution of this agent in some other way. The same applies to methods for cleaning all materials other than textiles, in particular hard surfaces. All conceivable washing or cleaning methods can be enhanced in at least one of the method steps by the use of a washing or cleaning agent as contemplated herein or an alpha-amylase as contemplated herein, and then constitute embodiments of the present disclosure. All elements, subjects and embodiments that are described for alpha-amylases as contemplated herein and agents that contain them can also be applied to this subject of the present disclosure. Therefore, at this juncture, reference is explicitly made to the disclosure at the corresponding point when it was indicated that this disclosure also applies to the above methods as contemplated herein.

Since alpha-amylases as contemplated herein naturally already have hydrolytic activity and these also develop in media that otherwise have no cleaning force, such as in simple buffers, an individual and/or the only step of a method of this kind can include, if desired, in bringing an alpha-amylase as contemplated herein into contact with the stain as the only component that has a cleaning effect, preferably in a buffer solution or in water. This constitutes a further embodiment of this subject of the present disclosure.

Methods for treating textile raw materials or for textile care in which an alpha-amylase as contemplated herein becomes active in at least one method step also constitute alternative embodiments of this subject of the present disclosure. Of such methods, methods for textile raw materials, fibers or textiles having natural components are preferred, and very particularly for those containing wool or silk.

All elements, subjects and embodiments that are described for alpha-amylases as contemplated herein and agents that contain them can also be applied to this subject of the present disclosure. Therefore, at this juncture, reference is explicitly made to the disclosure at the corresponding point when it was indicated that this disclosure also applies to the above use as contemplated herein.

In a further aspect, the present disclosure relates to the use of an alpha-amylase as contemplated herein or of an alpha-amylase that can be obtained using a method as contemplated herein in a washing or cleaning agent for removing starch-containing stains. All elements, subjects and embodiments that are described for alpha-amylases as contemplated herein and agents that contain them can also be applied to this subject of the present disclosure.

### EXAMPLES

### Example 1

### Brief Summary of the Experiment Process

In a screening, basidiomycetes were specifically screened for enzymes which break down starch. Here, a wild-type enzyme from Rhizoctonia solani was found which demonstrates good washing performance on various starch-containing textiles. The enzyme found is annotated as an alpha-amylase.

The sequence of the protein found differs significantly from the sequences of amylases which have previously been used in L&HC. The enzyme therefore provides many opportunities for increasing the genetic and biochemical variety in the range of amylases used in cleaning agents.

Washing Agent Matrix Used

This is the washing agent matrix (which is commercially available, without enzymes, optical brighteners, perfume and dyes) used for the washing test:

| | | wt. % of |
|---|---|---|
| | wt. % of active substance | active substance |
| Chemical name | in the raw material | in the formulation |
| Water demineralized | 100 | Remainder |
| Alkylbenzene sulfonic acid | 96 | 4.4 |
| Anionic surfactants | 70 | 5.6 |
| C₁₂-C₁₈ fatty acid Na salt | 30 | 2.4 |
| Non-ionic surfactants | 100 | 4.4 |
| Phosphonates | 40 | 0.2 |
| Citric acid | 100 | 1.4 |
| NaOH | 50 | 0.95 |
| Defoamers | t.q. | 0.01 |
| Glycerol | 100 | 2 |
| Preservatives | 100 | 0.08 |
| Ethanol | 93 | 1 |

Without optical brighteners, perfume, dye and enzymes.
Dosage 4.7 g/L

Activity Assay

In order to determine the amylolytic activity of amylases as contemplated herein, a modified para-nitrophenyl maltoheptaoside was used, the terminal glucose unit of which was blocked by a benzylidene group. Para-nitrophenyl oligosaccharide is released from this molecule by the amylase, which para-nitrophenyl oligosaccharide is in turn converted into glucose and para-nitrophenol by employing the enzymes glucoamylase and alpha-glucosidase. The amount of released para-nitrophenol is thus proportional to the activity of the amylase. The measurement is taken for example using the Quick-Start® Test Kit from the company Abbott (Abbott Park, Ill., USA). The increase in absorption (405 nm) in the test batch was determined at 37° C. for 3 minutes against a photometric control value (blind value). The calibration was carried out against an enzyme standard having known activity (e.g. Maxamyl®/Purastar® 2900 Genencor 2900 TAU/g). The evaluation is made by determining the absorption difference dE (405 nm) per minute against the enzyme concentration of the standard.

Washing Test and Results

A washing test was carried out using the purified supernatant from Rhizoctonia solani AG-3 which contains the wild-type alpha-amylase as contemplated herein.

Conditions: 40° C., 16° dH water, 1 h

Enzyme concentration: 0.18 TAU/ml (determining the amylase activity using benzylidene-blocked para-nitrophenol maltoheptaoside); this corresponds to an amount of amylase conventionally used in washing agents.

Stains:

| | |
|---|---|
| 1. C-S-26 | Maize starch |
| 2. C-S-27 | Potato starch |
| 3. C-S-28 | Rice starch |
| 4. C-S-29 | Tapioca starch |

A woven fabric blank (diameter=10 mm) was placed in a microtiter plate, washing liquor was preheated to 40° C., and the end concentration was 4.7 g/L;
the liquor and enzyme were put on the stain and incubated for 1 h at 40° C. and 600 rpm;
the stain was then rinsed several times with clear water and left to dry, and the lightness was determined using a color measurement device.

The lighter the woven fabric, the better the cleaning performance. What was measured in this case was the L value=lightness, and the higher the value the lighter the stain.

Washing was carried out using a conventional liquid washing agent without any enzymes.

Sample 1: Washing agent without amylase as a benchmark (comparison reference)

Sample 2: Washing agent plus alpha-amylase from Rhizoctonia solani (as contemplated herein)

Result (from IP 2398):

| Stain | Sample 1 | Sample 2 |
|---|---|---|
| Maize starch | 76.8 | 82.8 |
| Potato starch | 76.2 | 82.7 |
| Rice starch | 76.7 | 81.4 |
| Tapioca starch | 76.1 | 83.2 |

It is clear that the amylase performs well on all four stains. 1 unit is used to indicate a significant improvement in performance; in this case, up to 7.1 units of improvement have been achieved. As a negative control, the boiled purified supernatant from the production organism Rhizoctonia solani was also washed (99° C. for 30 minutes), and said supernatant does not demonstrate any washing performance.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An alpha-amylase comprising an amino-acid sequence which is at least about 70% identical to the amino-acid sequence shown in SEQ ID NO: 1 over the total length thereof.

2. An alpha-amylase obtained from an alpha-amylase according to claim 1 acting as a starting molecule by employing one or more conservative amino-acid substitutions.

3. A method for preparing an alpha-amylase according to claim 1, comprising providing a starting alpha-amylase which is at least about 70% identical to the amino-acid sequence shown in SEQ ID NO: 1 over the total length thereof.

4. The method according to claim 3, further comprising one or more of the following method steps:
(a) introducing one or more conservative amino-acid substitutions;
(b) altering the amino-acid sequence by means of fragmentation, deletion, insertion or substitution mutagenesis such that the alpha-amylase comprises an amino-acid sequence which matches the starting molecule over a length of at least about 360 interconnected amino acids.

5. The alpha-amylase of claim 1 formed from a nucleic acid.

6. The alpha-amylase of claim 1 formed from a vector comprising a nucleic acid.

7. (canceled)

8. The method of claim 3 comprising
(a) cultivating a host cell; and
(b) isolating the alpha-amylase from the host cell.

9. (canceled)

10. (canceled)

11. (canceled)

12. The alpha-amylase of claim 1 comprising an amino-acid sequence which is at least about 95% identical to the amino-acid sequence shown in SEQ ID NO:1 over the total length thereof.

13. The alpha-amylase of claim 1 comprising an amino-acid sequence which is at least about 99.8% identical to the amino-acid sequence shown in SEQ ID NO:1 over the total length thereof.

14. The alpha-amylase of claim 1 that is a free-enzyme.

15. The alpha-amylase of claim 1 that is additionally stabilized by being coupled to a polymer.

16. The alpha-amylase of claim 1 that has at least about 95% of the washing performance of an alpha-amylase which has an amino-acid sequence that corresponds to the amino-acid sequence shown in SEQ ID NO: 1.

17. The alpha-amylase of claim 2 obtained from an alpha-amylase comprising an amino-acid sequence which is at least about 70% identical to the amino-acid sequence shown in SEQ ID NO:1 over the total length thereof which acts as a starting molecule by means of fragmentation, deletion, insertion or substitution mutagenesis, and comprises an amino-acid sequence which matches the starting molecule over a length of at least about 360 interconnected amino acids.

18. The alpha-amylase of claim 2 obtained from an alpha-amylase comprising an amino-acid sequence which is at least about 99.8% identical to the amino-acid sequence shown in SEQ ID NO:1 over the total length thereof which acts as a starting molecule by means of fragmentation, deletion, insertion or substitution mutagenesis, and comprises an amino-acid sequence which matches the starting molecule over a length of at least about 360 interconnected amino acids.

19. The alpha-amylase of claim 2 obtained from an alpha-amylase comprising an amino-acid sequence which is at least about 70% identical to the amino-acid sequence shown in SEQ ID NO:1 over the total length thereof which acts as a starting molecule by means of fragmentation, deletion, insertion or substitution mutagenesis, and comprises an amino-acid sequence which matches the starting molecule over a length of at least about 513 interconnected amino acids.

20. The alpha-amylase of claim 2 obtained from an alpha-amylase comprising an amino-acid sequence which is at least about 99.8% identical to the amino-acid sequence shown in SEQ ID NO:1 over the total length thereof which acts as a starting molecule by means of fragmentation, deletion, insertion or substitution mutagenesis, and comprises an amino-acid sequence which matches the starting molecule over a length of at least about 513 interconnected amino acids.

21. The method of claim 4 comprising both steps (a) and (b).

22. The alpha-amylase of claim 6 wherein the vector is a cloning vector.

23. The alpha-amylase of claim 6 wherein the vector is an expression vector.

24. The method of claim 8 wherein the host cell is a non-human host cell that secretes the alpha-amylase into a culture medium surrounding the non-human host cell.
